(19) United States
(12) Patent Application Publication
(10) Pub. No.: US 2023/0165472 A1
(43) Pub. Date: Jun. 1, 2023

(54) DEVICES, SYSTEMS AND METHODS FOR MONITORING PHYSIOLOGICAL CHARACTERISTICS OF A PATIENT

(71) Applicant: Vlepis Solutions Pty Ltd, Sydney (AU)

(72) Inventors: Fang CHEN, Epping (AU); Sharath SRIRAM, Docklands (AU); Bill DIMOPOULOS, Haberfield (AU)

(21) Appl. No.: 17/913,246

(22) PCT Filed: Mar. 25, 2021
(86) PCT No.: PCT/AU2021/050266

§ 371 Date: Sep. 21, 2022

(30) Foreign Application Priority Data
Mar. 25, 2020 (AU) 2020900903
Dec. 24, 2020 (AU) 2020904836

(51) Int. Cl. A61B 5/0205; A61B 5/00; A61B 5/026; A61B 5/11

(52) U.S. Cl. CPC A61B 5/0205; A61B 5/6833; A61B 5/002; A61B 5/026; A61B 5/1118; A61B 2560/0214; A61B 2560/0271; A61B 2562/06

(57) ABSTRACT

The present invention provides a device for monitoring a plurality of physiological characteristics of a patient. The present invention also relates to methods and systems for monitoring a plurality of physiological characteristics of a patient employing said device. The present invention further provides methods and systems wherein multiple devices are used for monitoring a plurality of physiological characteristics of patients.

### FIELD OF THE INVENTION

The present invention relates to devices, systems and methods for monitoring a plurality of physiological characteristics of a patient. More particularly, the present invention relates to devices for monitoring a plurality of physiological characteristics of a patient wherein said devices adhere to patient, preferably the skin surface of the patient. The devices according to the present invention may further function to dynamically calculate the risk associated with the patient in respect of a particular disease or indication. The present invention also relates to methods and systems utilizing the described devices.

### BACKGROUND OF THE INVENTION

Mammalian skin provides a range of physiological signals which can provide indications of medical conditions well before traditional systems present. Personalised and wearable monitoring systems attempt to make use of these physiological signals to assist in monitoring and diagnosis of patient.

Wearable monitoring devices are typically limited to a narrow range or number of physiological systems which can be monitored. As such, methods and system employing current wearable technology are usually only to be used in situations wherein a specific medical condition is to be monitored. As a result, systems and methods employing these devices are typically unsuitable for long term or sustained monitoring of elderly patients who may have variety of long term medical conditions and do not permit of a response to changes in the plurality of characteristics being monitored.

Current wearable monitoring devices typically provide discomfort to the patient, and coupled with the need for bulky and ridged parts, may often lead to a reduced compliance by the user. In addition, these devices typically require supplementary add-ons such as power sources which may further reduce the ease of use of such devices in systems and methods for monitoring patients over an extended period.

There exists a need to overcome, or at least alleviate, one or more of the difficulties or deficiencies associated with the prior art.

### SUMMARY OF THE INVENTION

In one aspect, the present invention provides a device for monitoring a plurality of physiological characteristics of a patient including:
a first biosensor layer including a biodata collection element for sensing the physiological characteristics; and
an adhesive layer for maintaining contact between the biosensor layer and the skin surface of the patient.

In one embodiment, the patient includes a human being or other mammal for which it is desired to measure physiological characteristics. More preferably, the desire to measure physiological characteristics arises because of the need to treat the human or other mammal for a medical condition and/or to monitor changes in the physiological characteristics of the patient, and dynamically calculate the risk associated with the patient in respect of a particular disease or indication, and where necessary, respond appropriately.

The term ‘patient’ as used herein may include, but is not limited to, an elderly individual, a immunocompromised individual, a health compromised individual, an individual with a suspected or undiagnosed medical condition, an athlete or any other individual wherein there is a need to measure one or more physiological characteristics of said individual.

In a preferred embodiment, the biosensor layer includes a layer or series of layers which house the biodata collection element(s).

In an embodiment, the biodata collection element includes a collection of sensors, electrodes or any other such sensing elements capable of receiving and responding to a signal or stimulus emanating from the human or other mammal.

In an embodiment, the adhesive layer includes a chemical composition capable of adhering to the skin surface of the patient.

In a preferred embodiment, the biodata collection element includes two or more sensors responsive to different physiological inputs. In a further preferred embodiment, the biodata collection element includes four to eight sensors responsive to different physiological inputs.

In an embodiment, the physiological inputs include signals or stimuli presented by the patient which are determinative of a physiological characteristic. For example, the signals or stimuli may include an electrical signal, electromagnetic radiation, a physical property, a temperature, a gas, a liquid, a protein, a hormone or other such biological factor.

In an embodiment, the physiological inputs may include signals or stimuli presented by the patient which may be determinative of a mental health characteristic. For example, the physiological inputs may indicate that patient is experiencing anxiety, depression, post-traumatic stress disorder (PTSD), mental stress or any other such mental health condition.

In a preferred embodiment, the device is powered remotely. In a further preferred embodiment, the device is powered by connection to a Wi-Fi, Bluetooth, cellular or low-power wide-area network power source. In a further preferred embodiment, the device includes an electromagnetic radiation receiver for receiving electromagnetic radiation and circuit for extracting power for the device from the received electromagnetic radiation.

In a further preferred embodiment the electromagnetic radiation is received from a Wi-Fi, Bluetooth, cellular or low-power wide-area network. In a further preferred embodiment the cellular network is a 4G or 5G cellular network. In a further preferred embodiment the cellular network is a 6G cellular network. In an alternatively preferred embodiment the low-power wide-area network is a long range wide-area network (LoRaWAN). In a further preferred embodiment the device is powered by any suitable low powered communication mechanisms designed for IOT (internet of things) networks.

In a further preferred embodiment, the device is powered by a wireless network and a secondary power source. In an embodiment the secondary power source is a battery power source. In a particularly preferred embodiment the secondary power source is rechargeable battery. In an alternatively preferred embodiment the secondary power source is a replaceable battery.

The secondary power source as referred to herein may include be a gel pack battery, lithium-ion battery or any other battery type capable of powering the device for monitoring a plurality of physiological characteristics of a patient.

In a preferred embodiment, the sensors are activated and/or deactivated remotely. In a further preferred embodiment the device includes a receiver for receiving a signal for controlling a circuit to activate and/or deactivate the sensors.

In a preferred embodiment, the physiological inputs include heart rate, blood pressure, pulse, SpO2, VO2 max, movement time, rest time, lay time, body temperature, electrical activity of the heart (ECG), measurement of wound recovery factors, measurement of skin surface stress factors, measurement of blood sugar levels, audio output and/or the geographic location of the patient. In a further preferred embodiment the physiological input may include changes to red blood cell levels in the patient. In a further preferred embodiment, the physiological inputs may include changes in the patient's thoracic region, including patient mobility, movement, muscle contraction and/or strain. For example, monitoring the patient's thoracic region for said physiological inputs may be used to monitor voice detection.

In a further preferred embodiment, the physiological inputs may include movement, movement rate and/or accelerometer inputs exhibited by the patient.

In a further preferred embodiment, the physiological inputs may include perfusion within the monitored region of the patient.

In a further preferred embodiment, the physiological inputs are signals or stimuli presented by the patient which are determinative of a mental health characteristic.

By ‘SpO2’ as used herein is meant peripheral oxygen saturation, an estimation of haemoglobin oxygen saturation levels observed within the patient.

By ‘VO2 max’ as used herein is meant the maximum rate of oxygen consumption of a patient, as measured during increasing physical stress or exercise.

By the term ‘movement time’ as used herein, is meant the amount of time the patient spends moving in same movement activity.

By the term ‘rest time’, as used herein, is meant the amount of time the patient spends resting in the same rest between performing activities.

By the term ‘lay time’, as used herein, is meant the amount of time the patient spends laying down in the same laying session. The ‘lay time’ may include, but is not limited to, any length of time wherein the patient is sleeping, resting, has experienced a fall or any other incident wherein the patient is in a non-upright position.

By ‘audio output’ as used herein is meant any audible noise expressed by the patient, or noise produced by the patient. For example, audio output may include monitoring for a word count spoken by the patient. In a further example, audio output may include monitoring for patient breathing rate and/or intensity.

By perfusion as used herein is meant the rate of passage for fluid through the patient's circulatory system or lymphatic system to an organ or a tissue.

In one embodiment, measurement of wound recovery factors includes recording levels of biological factors and/or recording physiological responses involved in wound recovery. Biological factors may include, but are not limited to, proteins, hormones, enzymes and/or platelets expressed as a result of wound recovery. Physiological responses may include, but are not limited to, blood clot formation, vascular constriction and/or physical contraction.

In a preferred embodiment, measurement of skin surface stress factors includes recording levels of biological factors and/or recording physiological responses involved in skin surface stress recovery. For example, one such biological factor which may be monitored is hormone expression or hormone level changes within the patient, an example of one such hormone is the steroid hormone cortisol.

In a preferred embodiment measurement of blood sugar levels includes recording blood glucose levels, blood ketone levels or any other such indicator of a change in the patient's blood sugar level.

In a preferred embodiment, the device includes a radio frequency transmitter, such as a Wi-Fi, Bluetooth, cellular or low-power wide-area network transmitter. In a further preferred embodiment the cellular network is a 4G or 5G cellular network. In a further preferred embodiment the cellular network is a 6G cellular network.

In a preferred embodiment the device is configured to allow for network triangulation in order to provide the pinpoint geographic position of the device. In a further preferred embodiment the device is configured to allow for network triangulation when the device is located within a cellular network or low-power network.

Network triangulation as referred to herein is meant a process by which the location of a radio frequency transmitter can be determined by measuring either the radial distance, or the direction, of the received signal from two or three different points.

In a preferred embodiment the biodata collection element is configured to record the physiological inputs. In a further preferred embodiment the transmitter is configured to transfer the recorded physiological inputs to a database for storage. In a particularly preferred embodiment the recorded physiological inputs are transferred to the database by using the a Wi-Fi, Bluetooth, cellular or low-power wide-area network transmitter. In a further preferred embodiment the cellular network is a 4G or 5G cellular network. In a further preferred embodiment the cellular network is a 6G cellular network. In a further preferred embodiment the recorded physiological inputs are transferred to the database by using any suitable low powered communication mechanisms designed for IOT (internet of things) networks.

By the term ‘transmitter’ is meant any suitable element capable of transferring the recorded physiological inputs to a database for storage. For example said transmitter may include a removable SIM card, eSIM, iSIM or any suitable silicon wafer capable of transferring the recorded physiological inputs to a database for storage.

In a preferred embodiment the database is on a remote server. In a more preferred embodiment the remote server includes a dedicated server or a virtual server.

By the term ‘remote server’, as used herein, is meant a server that is remotely located and connected by computer network.

In an embodiment the server is configured to interpret the plurality of physiological inputs and/or to monitor for changes in the plurality of physiological inputs.

In a preferred embodiment the device includes a protective outer layer.

In a preferred embodiment the device includes a machine readable tag for assigning identification details of the patient to the device. In a more preferred embodiment the machine readable tag is a two dimensional barcode. In a particularly preferred embodiment the two dimensional barcode is a quick response (QR) barcode. In a preferred embodiment, the machine readable tag is a near field identifier, such as a RFID tag.

By the term ‘identification details’, as used herein, is meant details to aid in identifying the patient which may be linked to the recorded physiological inputs. Examples of identification details may include, but are not limited to, patient name, gender, age, birth date, known medical conditions, medical history, ongoing treatments or medicine/drugs currently being administered to the patent.

In a preferred embodiment, the device is formed from flexible materials.

In a preferred embodiment the adhesive layer is resistant to damage from sweat and/or water. In an alternatively preferred embodiment the adhesive layer is resistant to causing irritation or damage to the patient.

In a preferred embodiment the adhesive layer includes an adhesive selected to minimise irritation or damage to the skin of the patient, whilst maintaining functional contact with the skin for an extended period.

By the term ‘an extended period’, as used herein is meant approximately between 1 to 30 days, more preferably between 1 to 14 days, and more preferably between 1 to 10 days.

In a preferred embodiment the adhesive layer maintains contact with the skin surface of the user for between approximately 1 to 30 days. In a more preferred embodiment the adhesive layer is capable of maintaining contact with the skin surface of the user for between approximately 1 to 14 days. In a particularly preferred embodiment the adhesive layer is capable of maintaining contact skin surface of the user for between approximately 1 to 10 days.

In a preferred embodiment, the device includes a visual means for indicating the status of the device. In a more preferred embodiment the visual means includes a colour change indicator. In a more preferred embodiment the status of the device is selected from activated, deactivated, idle and due for replacement.

In an embodiment the colour change indicator includes a suitable element capable of changing colour based on a change is status of the device, thereby indicating to the patient or other the status of the device.

The device as described herein may include dielectric resonator-based, mechanically tunable, metasurface design elements, such as those disclosed in Gutruf et al. (2016), the entirety of which is incorporated by reference.

The device as described herein may include multifunctional metal oxide (zinc oxide, ZnO) based sensors on a prevalent, biocompatible, flexible substrate, polydimethylsiloxane (PDMS), such as is disclosed in Gutruf et al. (2015); the entirety of which is incorporated by reference.

The device as described herein may include devices that show minimal alteration in resistance when being subjected to strain; in particular devices including prevalent material combinations of gold as a conductor and polyimide as the flexible substrate, such as those disclosed in Gutruf et al. (2014); the entirety of which is incorporated by reference.

The device as described herein may include high-temperature-processed functional oxides with a flexible, elastomeric substrate; in particular devices with indium tin oxide (ITO) on polydimethylsiloxane (PDMS), such as is disclosed in Gutruf et al (2013); the entirety of which is incorporated by reference.

The device as described herein may include flexible or stretchable sensors for use in detecting a substance and/or electromagnetic radiation, wherein said devices may also include polymer isolation layer(s), as disclosed in the Australian patent 2016203718; the entirety of which is incorporated by reference. The devices described herein may include sensors including oxygen deficient metal oxide layer(s) such as zinc oxide (ZnO), indium tin oxide (ITO), tin oxide and/or titanium dioxide. The devices described herein may include polymers/elastomers including polyimide (PI), polymethyl methacrylate (PMMA), photo-patternable epoxy resin, polyethylene terephthalate (PET) and/or polydimethylsiloxane (PDMS).

In a second aspect of the present invention there is provided a method for monitoring the physiological characteristics of a patient including:
applying to the patient a device for sensing a plurality of physiological inputs, the device including:
   a first biosensor layer including a biodata collection element; and
   an adhesive layer;
wherein the adhesive layer provides a means for maintaining contact between the biosensor layer and the skin surface of the patient; and
wherein upon activation the device senses one or more physiological inputs from the patient.

In a preferred embodiment the biodata collection element is responsive to different physiological inputs. In a particularly preferred embodiment the one or more physiological inputs includes four to eight different physiological inputs.

In a preferred embodiment the method includes remotely powering the device. In a particularly preferred embodiment the device is powered by connection to a Wi-Fi, Bluetooth, cellular or low-power wide-area network power source. In a further preferred embodiment the cellular network is a 4G or 5G cellular network. In a further preferred embodiment the cellular network is a 6G cellular network. In a further preferred embodiment the device is powered by any suitable low powered communication mechanisms designed for IOT (internet of things) networks.

In a further preferred embodiment, the method includes use of a device powered by a wireless network and a secondary power source. In an embodiment the secondary power source is a battery power source. In a particularly preferred embodiment the secondary power source is rechargeable battery. In an alternatively preferred embodiment the secondary power source is a replaceable battery.

The secondary power source as referred to herein may include be a gel pack battery, lithium-ion battery or any other battery type capable of powering the device for monitoring a plurality of physiological characteristics of a patient.

In a preferred embodiment the recorded physiological inputs are selected from heart rate, blood pressure, pulse, SpO2, VO2 max, movement time, rest time, lay time, body temperature, electrical activity of the heart (ECG), measurement of wound recovery factors, measurement of skin surface stress factors, measurement of blood sugar levels, audio output and/or the geographic location of the patient.

In a preferred embodiment the recorded physiological inputs are transferred to a database. In a particularly preferred embodiment the recorded physiological inputs are transferred to a database by a Wi-Fi, Bluetooth, cellular or low-power wide-area network connection.

In a preferred embodiment the method includes use of a device configured to allow for network triangulation in order to provide the pinpoint geographic position of the device. In a further preferred embodiment the device is configured to allow for network triangulation when the device is located within a cellular network or low-power network.

In a preferred embodiment the database is stored on a remote server.

In a preferred embodiment the method further includes notifying a secondary device of the recorded physiological inputs. The secondary device may function to dynamically calculate the risk associated with the patient in respect of a particular disease or indication, and where necessary, alert a medical professional to respond appropriately.

In a preferred embodiment, the method includes determining whether the physiological input(s) reach a predefined threshold. When the threshold is reached, a notification is sent to a secondary device.

In a preferred embodiment the secondary device is selected from a computer, smart phone, tablet, touch screen device or any other suitable “smart device”. In a further preferred embodiment the secondary device includes a data module configured to receive, monitor and/or analyse the physiological inputs recorded by the first biosensor layer.

By a ‘data module’ as used herein is meant a computer program or application which allows the user to view, analyse and interact with the recorded physiological characteristics.

In a preferred embodiment the data module is configured to provide an output of the recorded physiological inputs.

In a preferred embodiment the recorded physiological inputs are output from the secondary device. In a preferred embodiment the output includes a recording of the physiological inputs at pre-determined times. In a further preferred embodiment the output includes recommendation(s) for treatment and/or intervention in respect of the patient.

According to a further embodiment of the invention, there is provided a computer program including instructions for controlling the device to operate according to any one of the methods defined above.

In a further aspect of the present invention there is provided a method for monitoring the physiological characteristics of a patient including:
applying to the patient two or more devices for sensing a plurality of physiological inputs, wherein each device includes:
   a first biosensor layer including a biodata collection element; and
   an adhesive layer;
wherein the adhesive layer provides a means for maintaining contact between the biosensor layer and the skin surface of the patient; and
wherein upon activation each device senses one or more physiological inputs from the patient.

In a preferred embodiment the method includes applying to the patient between one to five devices for sensing a plurality of physiological inputs. In a further preferred embodiment the method includes applying to the patient two to three devices for sensing a plurality of physiological inputs.

In a preferred embodiment the method includes applying to the patient at least one device for sensing a plurality of physiological inputs, wherein said inputs are signals or stimuli presented by the patient which are determinative of a mental health characteristic.

In a further aspect of the present invention, there is provided a system for monitoring the physiological characteristics of a patient including:
a device for recording a plurality of physiological inputs, the device including:
   a first biosensor layer including a biodata collection element for sensing the physiological characteristics; and
   an adhesive layer for maintaining contact between the biosensor layer and the skin surface of the patient;
a server for receiving the recorded physiological inputs from the device and for and storing the received physiological inputs; and
a secondary device for accessing the stored physiological inputs.

In a preferred embodiment the system includes between one to five devices for sensing a plurality of physiological inputs. In a further preferred embodiment the system includes two to three devices for sensing a plurality of physiological inputs.

In a preferred embodiment the method includes applying to the patient at least one device for sensing a plurality of physiological inputs, wherein said inputs are signals or stimuli presented by the patient which are determinative of a mental health characteristic.

In a preferred embodiment the biodata collection element includes two or more sensors responsive to different physiological inputs. In a particularly preferred embodiment the biodata collection element includes four to eight sensors responsive to different physiological inputs.

In a preferred embodiment the system further includes a wireless network. In an embodiment the device is powered remotely by the wireless network. In a particularly preferred embodiment the wireless network is a Wi-Fi, Bluetooth, cellular or low-power wide-area network. In an embodiment the device is powered by connection to the Wi-Fi, Bluetooth, cellular or low-power wide-area network. In a further preferred embodiment the cellular network is a 4G or 5G cellular network. In a further preferred embodiment the cellular network is a 6G cellular network. In a further preferred embodiment the device is powered by any suitable low powered communication mechanisms designed for IOT (internet of things) networks.

In a further preferred embodiment, the device is powered by a wireless network and a secondary power source. In an embodiment the secondary power source is a battery power source. In a particularly preferred embodiment the secondary power source is rechargeable battery. In an alternatively preferred embodiment the secondary power source is a replaceable battery.

The secondary power source as referred to herein may include be a gel pack battery, lithium-ion battery or any other battery type capable of powering the device for monitoring a plurality of physiological characteristics of a patient.

In a preferred embodiment the recorded physiological inputs include heart rate, blood pressure, pulse, SpO2, VO2 max, movement time, rest time, lay time, body temperature, electrical activity of the heart (ECG), measurement of wound recovery factors, measurement of skin surface stress factors, measurement of blood sugar levels, audio output and/or the geographic location of the patient.

In a preferred embodiment the recorded physiological inputs are stored in a database. In a further preferred embodiment the device is configured to transfer the recorded physiological inputs to a database by a Wi-Fi, Bluetooth, cellular or low-power wide-area network connection. In a further preferred embodiment the cellular network is a 4G or 5G cellular network. In a further preferred embodiment the cellular network is a 6G cellular network. In a further preferred embodiment the device is configured to transfer the recorded physiological inputs to a database by any suitable low powered communication mechanisms designed for IOT (internet of things) networks.

In a preferred embodiment the system includes a device configured to allow for network triangulation in order to provide the pinpoint geographic position of the device. In a further preferred embodiment the device is configured to allow for network triangulation when the device is located within a particular network or low-power network.

In a preferred embodiment the database is located on a remote server. In a further preferred embodiment the remote server includes a dedicated server or a virtual server.

In a preferred embodiment the system includes a means for notifying the secondary device of the recorded physiological inputs. In a further preferred embodiment, the server is configured to detect when the recorded physiological input(s) reach a predefined threshold. In an embodiment when the threshold is reached a notification is sent to the secondary device.

In a preferred embodiment the secondary device is one of a computer, smart phone, tablet, touch screen device or any other suitable “smart device”.

In a preferred embodiment the secondary device includes a data module configured to receive, monitor and/or analyse the recorded physiological inputs. In a further preferred embodiment the data module is configured to provide an output of the recorded physiological inputs.

In a preferred embodiment the output includes the physiological inputs over pre-determined times. In a further preferred embodiment the output includes recommendation(s) for treatment and/or intervention in respect of the patient.

In a preferred embodiment the data module provides a means for identifying changes in the recorded physiological inputs.

In a preferred embodiment the system includes a means to record the patient interacting with a tertiary device.

In a preferred embodiment the ‘tertiary device’ includes a device capable of communicating the position of the patient relative to a device. The interaction with a device may be recorded in order to determine how often a patient wearing the device interacts with the device.

The term ‘tertiary device’, as used herein, is any such device capable of transmitting information to the device for monitoring a plurality of physiological characteristics of a patient. The tertiary device may be capable of communicating the position of the patient relative to a device. A tertiary device may include, but is not limited to, a ‘Smart Webster pack’ which is able to record and transmit information regarding a patient's interaction with the ‘Smart Webster pack’. A further example of a tertiary device includes a RFID tagged assisted walking device or any other device used by the patient. A further example of a tertiary device includes an environmental sensor which monitors room temperature, airflow or the opening/closing of a door within the environment.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

In the Figures:

FIG. 1 shows a device for monitoring a plurality of physiological characteristics, according to an embodiment of the present invention.

FIG. 2 shows an example of the device for monitoring a plurality of physiological characteristics in use by a patient.

FIG. 3 shows a work flow for monitoring a plurality of physiological characteristics according to an embodiment of the present invention.

FIG. 4 shows a block diagram of a system for monitoring a plurality of physiological characteristics according to an embodiment of the present invention.

FIG. 5 shows an example of multiple devices in use for monitoring a plurality of physiological characteristics in use by a patient. The devices shown include sensing for physiological characteristic indicative of both physiological and mental health characteristics, and transmission of said inputs to database storage.

### DETAILED DESCRIPTION OF THE EMBODIMENTS

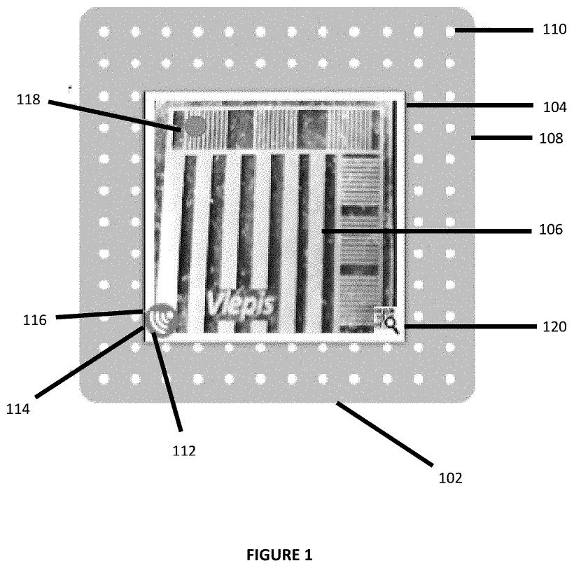

Referring to FIG. 1, there is provided a diagram of a device for monitoring a plurality of physiological characteristics (102) according to an aspect of the present invention. The device includes a first biosensor layer (104) which further includes a biodata collection element (106) for sensing for sensing the physiological characteristics. In an embodiment the device includes an adhesive layer (108) and a chemical composition (110) for adhering to the skin surface of a patient. In an embodiment the device also includes an electromagnetic radiation receiver (112) and electromagnetic radiation circuits (114) to allow for the device to be powered by a Wi-Fi, Bluetooth, cellular or low-power wide-area network electromagnetic radiation. In an embodiment the device includes a radiofrequency transmitter (116) to allow for sensed and recorded signals to be transferred to a remote server. In an embodiment the device includes a colour change indicator (118) to communicate the status of the device, and a machine readable tag (120) for assigning identification details of the patient to the device.

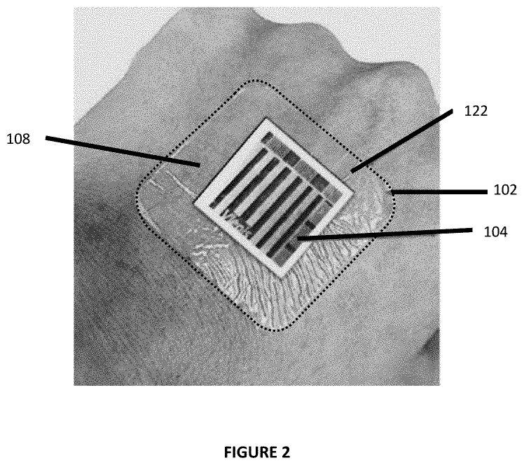

FIG. 2 shows an embodiment of the device for monitoring a plurality of physiological characteristics (102) in use by a patient, wherein the device s a first biosensor layer (104) which is held in contact with the skin of a patient by an adhesive layer (108). The device further includes a protective outer layer (122) covering the device.

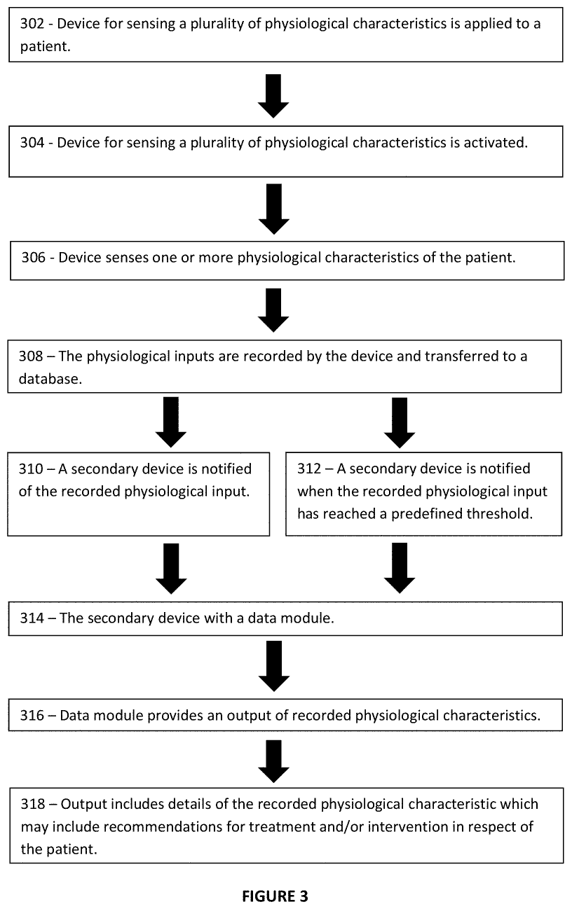

Referring to FIG. 3, there is provided a work flow of a method for monitoring a plurality of physiological characteristics, according to an aspect of the present invention. In an embodiment the device for monitoring a plurality of physiological characteristics is applied to the skin of a patient (302), the device is then activated (304) and upon activation the device senses and records one or more physiological inputs of the patient (306). The recorded physiological inputs are transferred to a database (308) where they are stored. Next a secondary device is notified either of the recorded physiological input (310) or notified upon the physiological input reaching a predefined threshold (312). The secondary device notified a data module configured to receive, monitor and/or analyse the physiological inputs (314), wherein the data module provides an output of the recorded physiological characteristics (316), and/or a recommended treatment or response to the recorded physiological characteristics (318).

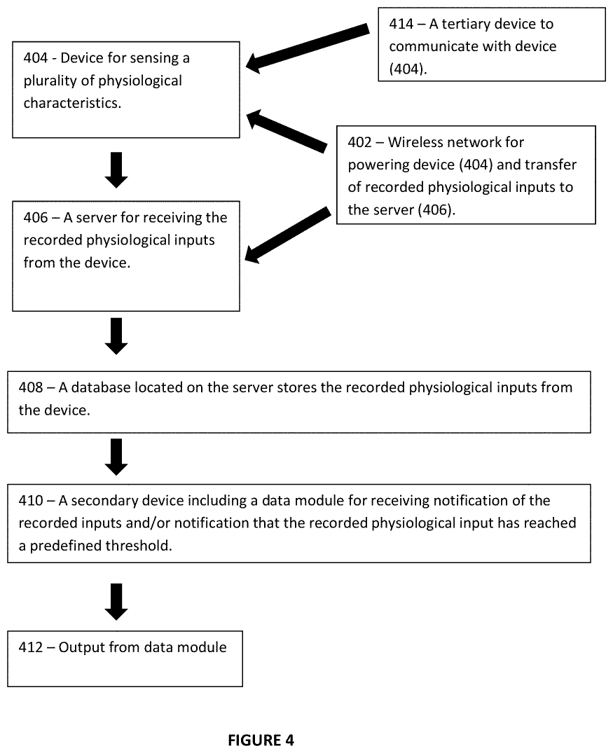

Referring to FIG. 4, there is provided the features of a system for monitoring a plurality of physiological characteristics, according to an aspect of the present invention. In an embodiment the system includes a device for sensing a plurality of physiological inputs.(404), wherein the device is powered by connection to a wireless network (402), such as a Wi-Fi, Bluetooth, cellular or low-power wide-area network. In an embodiment the system includes the device (404) which senses and records physiological inputs of a patent which are then transferred to a server (406) and stored in a database (408). In an embodiment the system includes a secondary device further including a data module (410) which receives notification of the recorded inputs and/or notification that the recorded physiological input has reached a predefined threshold. In an embodiment the data module provides an output relating to the recorded physiological input (412). In an embodiment the system also includes a tertiary device (414) which communicates with the device (404) to provide details relating to the position of the patient relative to a device (404).

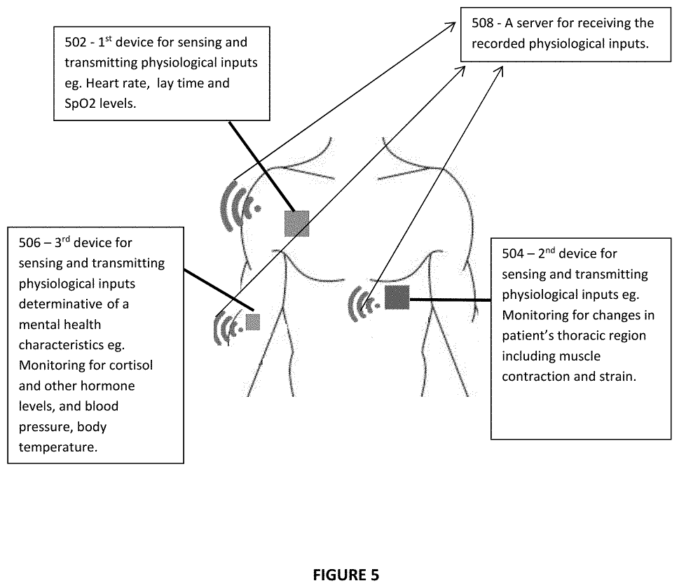

Referring to FIG. 5 there is provided the features of a system for monitoring a plurality of or monitoring a plurality of physiological characteristics through use of multiple devices, according to an aspect of the present invention. In an embodiment the method and system includes sensing a plurality of physiological inputs indicative of a physiological condition (502), wherein said device senses the physiological inputs which are then transferred to a server (508). In an embodiment the system includes a secondary device for sensing and transfer of a plurality of physiological inputs indicative of a physiological condition (504). In an embodiment the system includes a third device for sensing and transfer of a plurality of physiological inputs, wherein said inputs are indicative of a mental health condition (506), wherein said device is used in conjunction with multiple other devices (502 and 504) for sensing a plurality of physiological inputs and transfer of said inputs to a server (508).

Finally, it is to be understood that various alterations, modifications and/or additions may be made without departing from the spirit of the present invention as outlined herein.

### REFERENCES

P. Gutruf, C. Zou, W. Withayachumnankul, M. Bhaskaran, S. Sriram, and C. Fumeaux, 2016: Mechanically Tunable Dielectric Resonator Metasurfaces at Visible Frequencies. ACS Nano 10: 133-141.
P. Gutruf, E. Zeller, S. Walia, H. Nili, S. Sriram and M. Bhaskaran, 2015: Stretchable and Tunable Microtectonic ZnO-Based Sensors and Photonics. Small 11, No. 35, 4532-4539.
P. Gutruf, S. Walia, M. N. Ali, S. Sriram, and M. Bhaskarana, 2014: Strain response of stretchable micro-electrodes: Controlling sensitivity with serpentine designs and encapsulation. Appl. Phys. Lett. 104, 021908, 1-4.
P. Gutruf, C. M. Shah, S. Walia, H. Nili, A. S. Zoolfakar, C. Karnutsch, K. Kalantar-zadeh, S. Sriram and M. Bhaskaran, 2013: Transparent functional oxide stretchable electronics: micro-tectonics enabled high strain electrodes. NPG Asia Materials 5, e62, 1-7.
S. Sriram, M. Bhaskaran and P. Gutruf, 2016: A FLEXIBLE OR STRETCHABLE SENSOR FOR USE IN DETECTING A SUBSTANCE AND/OR ELECTROMAGNETIC RADIATION AND A METHOD FOR DETECTION THEREOF. Australian Patent No. 2016203718.

1. A device for monitoring a plurality of physiological characteristics of a patient including:
a first biosensor layer including a biodata collection element for sensing the physiological characteristics; and
an adhesive layer for maintaining contact between the biosensor layer and the skin surface of the patient.

2. The device according to claim 1, wherein the biodata collection element includes two or more sensors responsive to different physiological inputs.

3. (canceled)

4. The device according to of claim 1, wherein the device is powered remotely.

5. (canceled)

6. (canceled)

7. (canceled)

8. (canceled)

9. The device according to claim 2, wherein the sensors are activated and deactivated remotely.

10. (canceled)

11. The device according to claim 2, wherein the physiological inputs include heart rate, blood pressure, pulse, SpO2, VO2 max, movement time, rest time, lay time, body temperature, electrical activity of the heart (ECG), measurement of wound recovery factors, measurement of skin surface stress factors, measurement of blood sugar levels, audio output and/or the geographic location of the patient.

12. (canceled)

13. The device according to claim 2 wherein the physiological inputs are recorded by the biodata collection element.

14. The device according to claim 2 wherein the device includes a radio frequency transmitter.

15. The device according to claim 13, wherein the recorded physiological inputs are transferred to a database.

16. (canceled)

17. (canceled)

18. (canceled)

19. (canceled)

20. (canceled)

21. (canceled)

22. (canceled)

23. (canceled)

24. The device according to claim 1, wherein the device is formed from flexible materials.

25. (canceled)

26. (canceled)

27. (canceled)

28. The device according to claim 1, wherein the adhesive layer maintains contact with the skin surface of the user for between approximately 1 to 30 days.

29. (canceled)

30. (canceled)

31. (canceled)

32. (canceled)

33. (canceled)

34. A method for monitoring the physiological characteristics of a patient including:
applying to the patient one or more devices for sensing a plurality of physiological inputs, the device including:
a first biosensor layer including a biodata collection element; and
an adhesive layer;
wherein the adhesive layer provides a means for maintaining contact between the biosensor layer and the skin surface of the patient; and
wherein upon activation the device senses one or more physiological inputs from the patient.

35. (canceled)

36. (canceled)

37. (canceled)

38. (canceled)

39. The method according to claim 33, wherein the biodata collection element includes two or more sensors responsive to different physiological inputs.

40. (canceled)

41. The method according to claim 34, wherein the method incudes remotely powering the device.

42. (canceled)

43. (canceled)

44. The method according to claim 34, wherein the recorded physiological inputs are selected from heart rate, blood pressure, pulse, SpO2, VO2 max, movement time, rest time, lay time, body temperature, electrical activity of the heart (ECG), measurement of wound recovery factors, measurement of skin surface stress factors, measurement of blood sugar levels, audio output and/or the geographic location of the patient.

45. (canceled)

46. (canceled)

47. (canceled)

48. (canceled)

49. The method according to claim 34, wherein the method includes notifying a secondary device of the recorded physiological inputs.

50. The method according to claim 34, wherein the method includes determining whether a change in the recorded physiological input(s) reaches a predefined threshold.

51. (canceled)

52. (canceled)

53. The method according to claim 49, wherein the secondary device includes a data module configured to receive, monitor and/or analyse the physiological inputs recorded by the first biosensor layer.

54. (canceled)

55. (canceled)

56. (canceled)

57. A system for monitoring the physiological characteristics of a patient including:
a device for recording a plurality of physiological inputs, the device including:
a first biosensor layer including a biodata collection element for sensing the physiological characteristics; and
an adhesive layer for maintaining contact between the biosensor layer and the skin surface of the patient; and
a server for receiving the recorded physiological inputs from the device and for and storing the received physiological inputs.

58. (canceled)

59. (canceled)

60. (canceled)

61. (canceled)

62. (canceled)

63. The system according to claim 57, wherein the system includes a wireless network; and the device is powered remotely by the wireless network.

64. (canceled)

65. (canceled)

66. (canceled)

67. (canceled)

68. (canceled)

69. (canceled)

70. (canceled)

71. (canceled)

72. (canceled)

73. The system according to claim 57, wherein the system includes a means for notifying the secondary device of the recorded physiological inputs.

74. (canceled)

75. (canceled)

76. (canceled)

77. (canceled)

78. (canceled)

79. (canceled)

80. (canceled)

81. (canceled)

82. (canceled)
